(19) United States
(12) Patent Application Publication
(10) Pub. No.: US 2012/0116327 A1
(43) Pub. Date: May 10, 2012

(54) SURGICAL CLEANSING SOLUTIONS

(75) Inventors: Daniel Calladine, Oxford (GB); Henry Barnabas Smith, Gloucestershire (GB)

(73) Assignee: OCULYNX LIMITED, London (GB)

(21) Appl. No.: 13/383,909

(22) PCT Filed: Jul. 14, 2010
(86) PCT No.: PCT/GB2010/001342

§ 371 Date: Jan. 13, 2012

(30) Foreign Application Priority Data
Jul. 14, 2009 (GB) 0912164.1
Mar. 19, 2010 (GB) 1004563.1

(51) Int. Cl. A61M 35/00; A61F 9/00

(52) U.S. Cl. 604/295; 604/289

(57) ABSTRACT

A sterile pack (10) contains a sealed sterile container (2) container having a single-use seal (6) capable of being opened manually by a user. The container contains a cleansing solution (4) at a concentration suitable for immediate use in direct contact with the skin, periocular tissue or ocular surface of a human or animal. A removable label may be provided for placement in a patient record to provide traceability.

This invention relates to cleansing solution for use in surgery and other medical procedures, particularly for cleaning an area of the body on which a procedure is to be performed.

In surgical theatres there is a continual need to clean skin prior to surgery in order to prevent infection of the wound. To take just a single example from the inventor's own field of practice, in ophthalmic surgery the skin, eyelids and eyelashes (hereafter known as periocular tissue), conjunctiva and cornea (hereafter known as ocular surface) are typically cleaned using a 5% povidone iodine aqueous solution in accordance with the specifications set out by the Royal College of Ophthalmology. The well-established procedure employed is to decant a more concentrated solution from a larger container into a small plastic pot, commonly known as a Gallipot, and then to dilute the solution to the required concentration using saline, again from a larger container. However, in diluting a more concentrated solution it is difficult to ensure exactly the right concentration of the povidone iodine solution is achieved in order to meet the recommended specifications, particularly as these specifications can change from time to time.

When viewed from a first aspect the present invention provides a sterile pack containing a sealed sterile container, said container having a single-use seal capable of being opened manually by a user and containing a cleansing solution at a concentration suitable for immediate use in direct contact with the skin, periocular tissue or ocular surface of a human or animal.

The invention also extends to packaging comprising a plurality of individually sealed sterile compartments, each compartment containing a sealed sterile container, said containers each having a single-use seal capable of being opened manually by a user and said containers each containing a cleansing solution at a concentration suitable for immediate use in direct contact with the skin, periocular tissue or ocular surface of a human or animal.

Thus it will be seen by those skilled in the art that in accordance with the invention a cleansing solution is provided in a pre-filled, sterile container which can be at exactly the required concentration and with the required constituents (e.g. buffering solutions) and which is available for immediate use without having to store separate larger containers and pour out and mix the solution. Moreover, since the containers are disposable, there is no need to rinse and sterilise a re-usable container. The sterile pack or packaging in accordance with the invention can also conveniently carry clear information regarding its contents, concentration and other information such as a use-by date.

The cleansing solution could be any suitable one depending on its intended use. In preferred embodiments the cleansing solution is aqueous or alcohol-based. To take one, non-limiting example, the solution could be an aqueous povidone iodine solution at concentration of 10% or less e.g. 5%, more particularly one in accordance with specifications from the Royal College of Ophthalmology. Other examples include an alcohol-based povidone iodine solution or aqueous or alcohol-based chlorhexidine. It should be appreciated however that the particular constitution of the cleansing solution is not essential to the invention. Moreover since in accordance with the invention the cleansing solution is pre-prepared and remains sealed and sterile until it is used, a stock of many different types and/or concentrations of solutions can be maintained far more practicably than if it were necessary to keep larger containers of more concentrated solutions for dilution as is the current practice. Indeed, there may be some substances which are used relatively infrequently where the concentrated solution reaches its use-by date after opening before it has all been used up thus leading to wastage.

The seal could take any convenient form but preferably comprises a removable lid sealed to the top of the container. The lid is preferably removable with a peeling action. Preferably a tab is provided on the lid to aid removal.

Such lid could be made of any suitable material. In some preferred embodiments the lid comprises a polyester film. One non-limiting example of a suitable lid material would be a 50 micron white polyester with a combi lacquer. This offers a number of advantages over other sealing options. This could be attached to the container by any suitable method e.g. ultrasonic welding or an adhesive, but the lid is preferably heat-sealed to the container. Preferably a bead-form heat seal is employed. This ensures that the lid is secure yet easy to peel. It also facilitates peeling without leaving loose remnants which could contaminate the cleaning solution.

In a set of preferred embodiments at least the inwardly-facing surface of the lid is of a material which acts to repel the contents of the container. Typically for example the lid is hydrophobic, preferably to both aqueous and alcohol-based solutions. This has been found to help prevent splashing when the lid is removed by avoiding droplets of liquid adhering to the underside of the lid.

The container itself could take any convenient form e.g. a round open pot (when the lid is removed) that resembles a conventional Gallipot. It may be made of any suitably chemically inert material. In preferred embodiments the container is made of a material allowing safe long-term storage of corrosive solutions such as povidone iodine and chlorhexidine. One non-limiting example of a suitable pot material would be polypropylene, e.g. touch clear polypropylene.

The container is preferably filled to no more than three quarters of its capacity, preferably no more than two thirds of its capacity, preferably approximately half its capacity. For example, the container may have a capacity of 60 to 70 ml and be filled with 30 to 40 ml of cleansing solution. This is sufficient for standard surgical techniques but helps to minimise the risk of spillage once open. The container could be made of any suitable material. One non-limiting example is polypropylene or a more chemically resistant plastic such as polyethylene terephthalate. The invention also extends to different sizes of container to suite different surgical procedures where more or less cleansing solution is required. For example, when cleaning a limb, the container may have a capacity of 180 to 200 ml and be filled with 100 to 120 ml of cleansing solution.

The sterile pack could take any convenient form. In one set of preferred embodiments, the pack comprises a sealed pocket containing the container. This could, for example, be formed from two sheets of material crimped, welded or otherwise bonded together to enclose a volume including the container. The interior of the pack could simply comprise air or could be filled with an inert gas or could be evacuated.

The pack could also contain other sterile materials or equipment to make a complete surgical pack suitable for a particular procedure or for general use. Alternatively a pack containing only the sterile container could itself be included within such a surgical pack.

In the packaging comprising a plurality of compartments, the compartments are preferably of the form of the sterile pack described above. The packaging could comprise for example a connected plurality of sealed pockets, each containing a container. One convenient example of this would be a quilted sheet or strip with e.g. perforations or other lines of weakness allowing separation of individual pockets from the rest of the sheet or strip. Alternatively, the compartments could be unconnected and simply provided together in a common box or stacked vertically in a gravity drop down box or other suitable container.

In a set of preferred embodiments, the container and/or sterile pack is provided with a removable label bearing identifying information—e.g. product details and a batch code. Ideally every component of the pack or container bears the batch code information to aid traceability. Such a label can be stuck into the records of the procedure for which the product was used. This level of traceability distinguishes such arrangements clearing from the prior art, particularly in skin cleansing products. It will be appreciated that it would simply not have been possible with the prior art technique of on-demand decanting and dilution.

A particular embodiment of the present invention will now be described, by way of example only, with reference to the accompanying drawings in which:

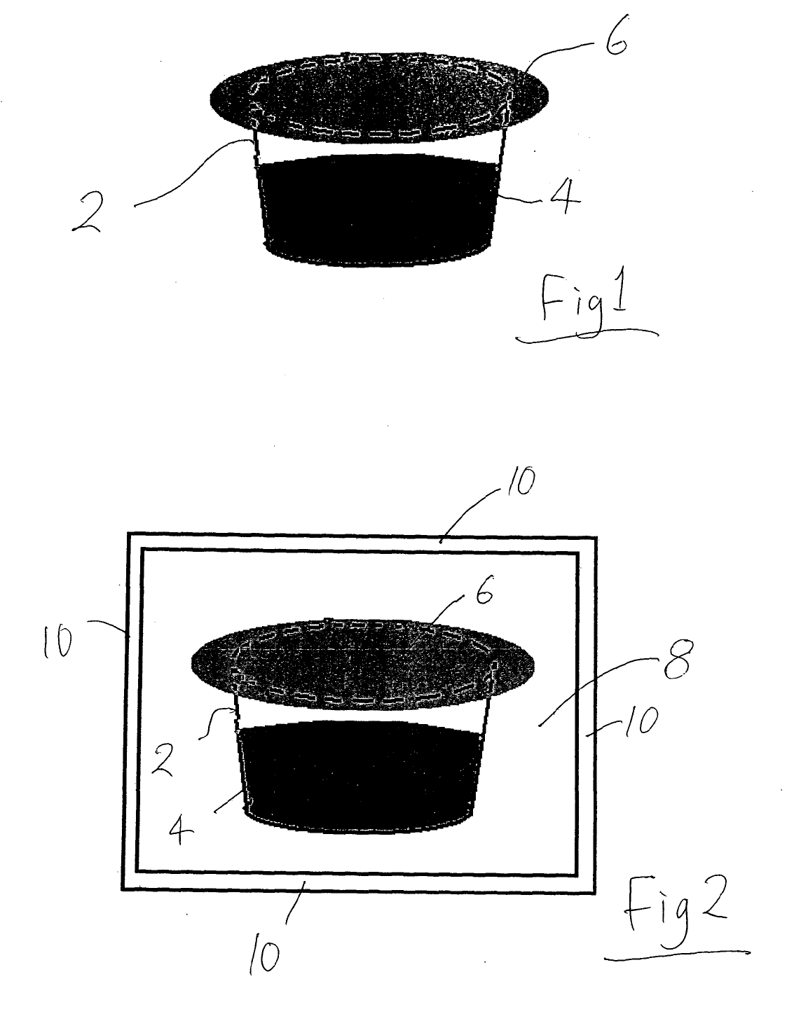

FIG. 1 is a schematic illustration of a container in accordance with the invention; and

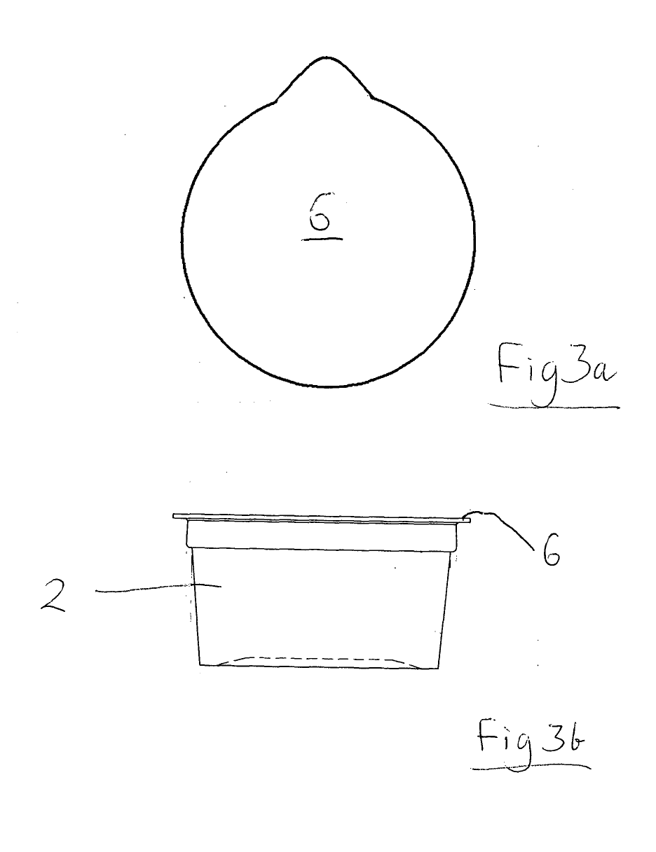

FIG. 2 is a schematic illustration of the container in a sterile pack.

FIG. 1 shows a round touch clear polypropylene pot 2 with a capacity of approximately 60 to 70 ml which contains 30 to 40 ml of a cleansing solution 4. The material of the pot 2 has undergone prolonged testing with 10% povidone iodine (PVP-I) and has shown itself to be both physically and chemically resistant with no discolouration. It is suitable for sterilisation by both gamma irradiation and ethylene oxide. It is suitable for heat-sealing to a number of different lid materials. The frosted finish offers some light protection whilst allowing the user to observe the content.

The pot 2 is sealed by a thin film lid 6 which could be made of a thin metal foil such as aluminium but is preferably a thin plastic film such as polyester—e.g. 50 micron white polyester with a combi lacquer . The lid 6 is heat-sealed to the upper rim of the pot 2 in order to provide a strong sealed connection to prevent leakage of the cleansing solution 4 but also to permit easy removal by peeling without tearing. The cleansing solution 4 could, for example, be a 5% solution of aqueous povidone iodine suitable for use in cleaning periocular tissue or the ocular surface prior to surgery.

Although not shown, the pot 2 is also provided with a detachable label bearing product details and a batch code.

FIG. 2 shows the pot 2 received in a pocket 8 which comprises two sheets bonded together around all four edges 10 in order to provide a sealed compartment. The pocket is suitably sterilised, e.g. irradiated with gamma rays in order to sterilise it and the container inside it. The pockets 8 could be supplied individually, as multiple individuals in boxes, or in a reel, strip or sheet of interconnected pockets but where the content of each pocket is a independently sterile. Batch codes may also be printed on the pockets or boxes.

The pot 2 is manufactured and packaged in a ‘clean room’ environment to reduce the risk of contamination. The pot 2 is then be subjected to sterilisation by irradiation or ethylene oxide to achieve sterility of the product within the outer packaging 8.

In use, a surgical assistant can take a pocket 8, open it up (a tear strip or line of weakness could be provided for this purpose) and remove the container 2. The batch label is then removed and placed on the records for the procedure to provide traceability. The lid 6 can then be removed by pulling it to break the seal around the rim of the pot 2. The cleansing solution 4 can then be used immediately without having to dilute it. The amount of cleansing solution 4 should be sufficient for all normal procedures, although if not, a second unit can be used. Any remaining solution can be discarded. Once the container has been used, it may also be discarded without need for sterilising and re-use.

In order to maintain a sterile surgical field the expected usage involves the pocket 8 being opened by a non-sterile assistant and the sealed container 2 being placed or dropped on to a sterile instrument table within the sterile field for use by the surgeon or surgical assistant who are suitably sterile. It is envisaged that variations of this technique will be common place; an example of an alternative technique is the non-sterile assistant half opening the pocket 8 and presenting it to the surgeon so that they can carefully remove the container 2 without touching the pocket 8. It is also envisaged that in some situations the pocket 8 may be packaged inside another pocket identical to pocket 8 but of a slightly larger size to provide a ‘double packaged’ product.

It will be seen that at least in accordance with the embodiment described above, a simple and convenient method of supplying cleansing solution is provided which does not rely on accurate judging of concentration or keeping stocks of concentrated solutions for subsequent dilution. It also ensures that sterility can easily be maintained. It will be appreciated however that the embodiment is simply one example of how the invention may be implemented and there are many possible variants within the scope of the attached claims.

1. A sterile pack containing a sealed sterile container, said container having a single-use seal capable of being opened manually by a user and containing a cleansing solution at a concentration suitable for immediate use in direct contact with the skin, periocular tissue or ocular surface of a human or animal.

2. A sterile pack as claimed in claim 1 wherein said seal comprises a removable lid sealed to the top of the container.

3. A sterile pack as claimed in claim 2 wherein the lid is removable with a peeling action.

4. A sterile pack as claimed in claim 2 wherein a tab is provided on the lid to aid removal.

5. A sterile pack as claimed in claim 2 wherein said lid is of polyester film.

6. A sterile pack as claimed in claim 2 wherein the lid is heat-sealed to the container.

7. A sterile pack as claimed in claim 1 comprising a label adapted to be detached for inclusion in a record, said label bearing identifying information for the pack.

8. A sterile pack as claimed in claim 1 wherein said container is filled to less than two-thirds of its maximum capacity with said cleansing solution.

9. A sterile pack as claimed in claim 1 wherein said cleansing solution is aqueous or alcohol-based.

10. A sterile pack as claimed in claim 1 wherein said cleansing solution comprises a povidone iodine solution.

11. A sterile pack as claimed in claim 10 wherein said solution has a povidone iodine concentration of 10% or less.

12. A sterile pack as claimed in claim 1 wherein said container has a capacity of between 60 and 70 ml.

13. A sterile pack as claimed in claim 1 comprising a sealed pocket containing the container.

14. Packaging comprising a plurality of individually sealed sterile compartments, each compartment containing a sealed sterile container, said containers each having a single-use seal capable of being opened manually by a user and said containers each containing a cleansing solution at a concentration suitable for immediate use in direct contact with the skin, periocular tissue or ocular surface of a human or animal.

15. (canceled)

16. Packaging as claimed in claim 1 comprising a connected plurality of sealed pockets, each containing a container.
